(19) United States
(12) Patent Application Publication
(10) Pub. No.: US 2026/0295132 A1
(43) Pub. Date: Oct. 1, 2026

(54) Adjustable Inner-Diameter Breast Pump Flange

(71) Applicants: Samira Jaafari Qawam, Santa Clara, CA (US); Jonathan Hodgkins, Chicago, IL (US); Theron Arden Hawley, San Francisco, CA (US)

(72) Inventors: Samira Jaafari Qawam, Santa Clara, CA (US); Jonathan Hodgkins, Chicago, IL (US); Theron Arden Hawley, San Francisco, CA (US)

(21) Appl. No.: 19/089,457

(22) Filed: Mar. 25, 2025

(51) Int. Cl. A61M 1/06

(52) U.S. Cl. CPC A61M 1/066

(57) ABSTRACT

An adjustable breast pump flange has an inner diameter that can be adjusted to accommodate various breast sizes without the need for additional inserts. The flange includes a collet mechanism that allows for precise diameter adjustment through a click-and-groove system. Constructed from soft, transparent silicone for enhanced comfort and visibility, the invention offers a customizable fit, improving the efficiency and comfort of milk expression for nursing mothers.

The materials filed herein contain no new matter, as required by 37 C.F.R. § 1.125(b). /RICHARD BENNETT SALLES/, Jul. 8, 2025 Adjustable Inner-Diameter Breast Pump Flange

### TECHNICAL FIELD

Technology for efficient breast pumps, breast pump adjustable fittings and collets, pump flanges, adjustable diameter collets and flanges tailored for breast pumps and for liquid-siphoning pumps, and associated components.

### JOINT CO-INVENTORS

SAMIRA JAAFARI QAWAM
JONATHAN HODGKINS
THERON ARDEN HAWLEY

### CLAIM OF PRIORITY

This application currently claims priority to, and incorporates by reference, no prior applications.

### BACKGROUND

A common issue with breast pump flanges is that their inner diameter is fixed, often resulting in a suboptimal fit for nursing mothers. Proper fit is essential for the efficient expression of breast milk, and a poorly fitting flange can lead to discomfort and reduced milk output. Existing solutions include silicone inserts that can be placed inside the flange to adjust the fit. However, these inserts are also fixed in size. As a nursing mother's breast size and shape naturally change throughout the postpartum period, multiple inserts of different sizes are often required, creating inconvenience and added expense.

This invention addresses the problem by incorporating an adjustable inner diameter directly into the breast pump flange itself. By integrating an adjustable mechanism, the flange can accommodate various breast sizes without the need for separate inserts, providing a more comfortable, efficient, and adaptable solution for nursing mothers.

### SUMMARY

The breast pump flange is constructed from soft, transparent silicone (in its ‘best mode’ embodiment) to comfortably contact the nursing mother's breast. The flange is designed in a substantially cone shape, with an adjustable inner diameter between about 17 mm and about 23 mm, allowing for a customized fit of nipple diameters between about 14 mm and about 20 mm.

The adjustable increments are, in the preferred embodiment, (in mm) 17, 18, 19, 20, 21, 22, and 23 mm. These numbers correspond respectively to nipple diameters of (in mm) 14, 15, 16, 17, 18, 19, and 20 mm. The adjustment mechanism features a durable plastic collet that utilizes a click-and-groove system, enabling precise diameter modifications between, inter alia, 17 mm and 23 mm. Each groove position will be hereafter referred to by its corresponding nipple diameter.

This innovative design eliminates the need for separate silicone inserts of fixed sizes. The opposite (distal) end of the cone connects seamlessly to the breast pump's milk collection bottle, ensuring efficient milk expression and convenience for the user.

### BRIEF DESCRIPTION OF THE DRAWINGS

FIG. 1 is an isometric view of an adjustable flange, in accordance with one embodiment of the present invention (with its flange removably-fixed in position 20).

FIG. 2 is an isometric view of the adjustable flange's aperture in process of closing, in accordance with one embodiment of the present invention (in position 15).

FIG. 3 is a side sectional view (isometric) of, inter alia, the button/collet/silicone funnel interaction positioning and mechanics (highlighting the main function, and unique design to accomplish this function, of the device), consistent with various embodiments;

FIG. 4 is a cut view of an adjustable flange, in accordance with one embodiment of the present invention in position 15. FIG. 4 therefore depicts a similar side sectional view (isometric) as that shown in figure three, herein the button/collet/silicone funnel interaction is shown moved to a new location/position, therein shrinking the aperture, consistent with various embodiments.

FIG. 5 is a Cut Side View: Flange assembly with Collet in 20 mm (the larger) diameter position. FIG. 5 is, therefore, a partial cut view of an adjustable flange, in accordance with one embodiment of the present invention (in position 20).

FIG. 6 is a Cut Side View: Flange assembly in 5 mm diameter (shrunk) position.

FIG. 7 is a partial cut view primarily showing the button detent engaged in the frame detent pocket. FIG. 7 therefore depicts a somewhat exploded view of the invention, showing captured silicone funnel, consistent with various embodiments.

### DEFINITIONS & REFERENCE NUMBERS

Cone (herein interchangeable with funnel)
12 Tube (image shows outer perspective of the invention's inner tube)
14 Tab
16 Opening (funnel opening)
20 Collet
22 Button A (sliding button #1 that moves along the groove on its side) 24 Button B (sliding
button #2 that moves along the groove on its side) Button Detent
28 Slot (herein interchangeable with groove) Frame A
32 Frame B
34 Frame Detent Pocket
36 Diameter Numbers engraved into frame for user's reference
40 Cap

### SPECIFICATIONS—DETAILED DESCRIPTION

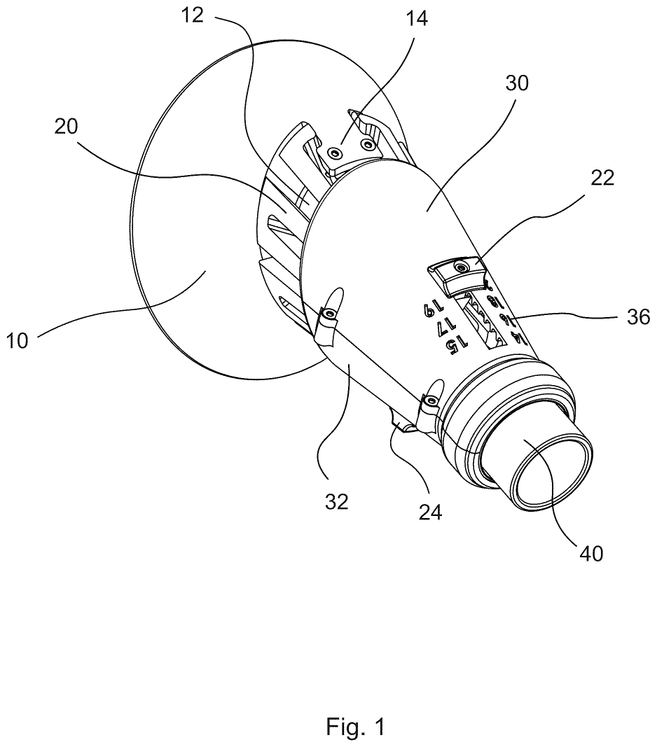

An implementation of the invention is a breast pump flange assembly for extracting breast milk with an adjustable fit for different nipple diameters. As shown in FIG. 1, the flange assembly is comprised primarily of a flexible rubber cone 10 and (inner) tube 12, a collet 20, a frame A 30, a frame B 32, a sliding button A 22, an alternate-side sliding button B 24, and a cap 40. The button A 22 and button B 24 are set by the user to different positions at the demarcated-(engraved) numbers 36 for user reference to adjust and remember the proper diameter for each individual breast and adjustment. For example, a 20 mm position, that may configure the flange assembly to fit a 20 mm nipple diameter, as shown in FIG. 1. The nipple diameter is measured at its base and does not include the areola.

An intended use case may involve attaching the cap 40 to a breast pump reservoir and placing the cone 10 onto a user's breast. The top cap 40 may be connected to any breast pump unit. The user can use either one (single pumping) or two (double pumping) flange assemblies with a breast pump.

The user can easily adjust the nipple diameter fit of the flange assembly by pressing and sliding button A 22 and button B 24. The button A and button B 24 can be used to adjust the collet 20 to different configurations suitable for nipple sizes that may include 20 mm, 19 mm, 18 mm, 17 mm, 16 mm, 15 mm and 14 mm.

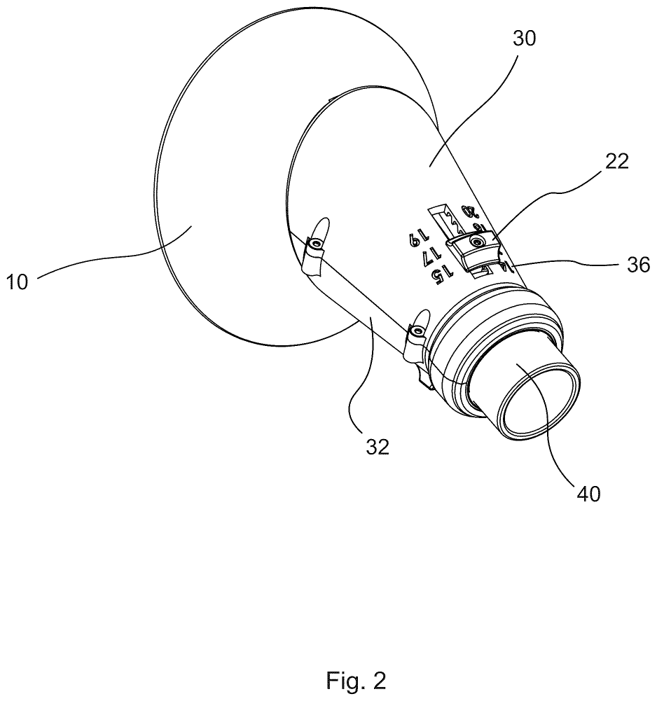

FIG. 2 shows the flange assembly with the collet 20 and the button A 22 in the 15 mm nipple diameter configuration.

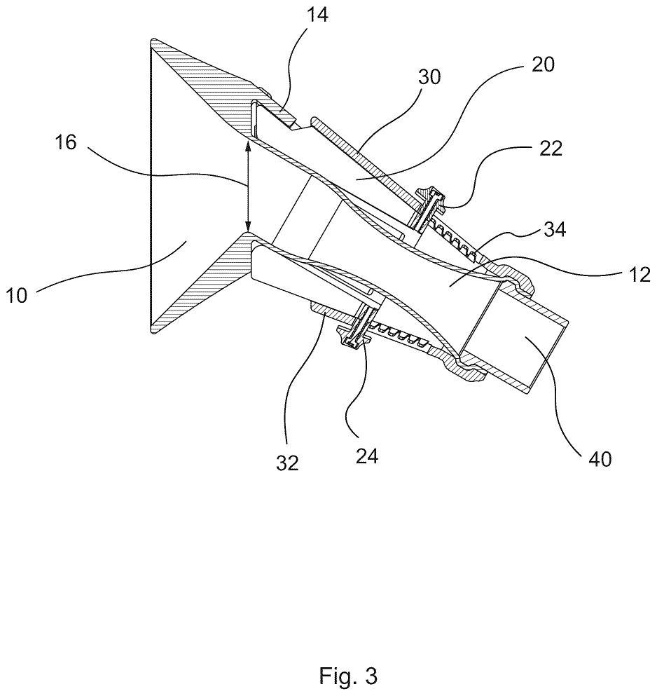

FIG. 3 is a side sectional view that shows the cone attached to the tube 12 at the opening 16.

The interior volume defined by the opening 16, the tube 12, and the cap 40 provide a passage for milk to flow into a breast pump reservoir. Preferably, the cone 10, the opening 16, and the tube 12 are made substantially of a flexible silicone material with a single smooth internal surface.

Rough surface texture or mold parting lines may aggravate the nipple tissue as it expands and contracts during pumping.

The opening 16 and the tube 12 are surrounded by the collet 20 which itself is surrounded by the frame A 30 and the frame B 32. The button A 22 and the button B 24 are permanently attached to the collet 20. Preferably, the tube 12 should be a flexible silicone material to allow the inner diameter of the collet 20 to radially compress the tube 12 at the opening 16 to set its diameter.

When the button A 22 and button B 24 are set to the 20 mm nipple diameter configuration the collet 20 may be set to an inner diameter that will cause the opening 16 to be 23 mm, as shown in FIG. 3. The additional 3 mm clearance between the opening 16 diameter and the diameter of the nipple helps prevent rubbing and irritation of the nipple as it expands and contracts during pumping.

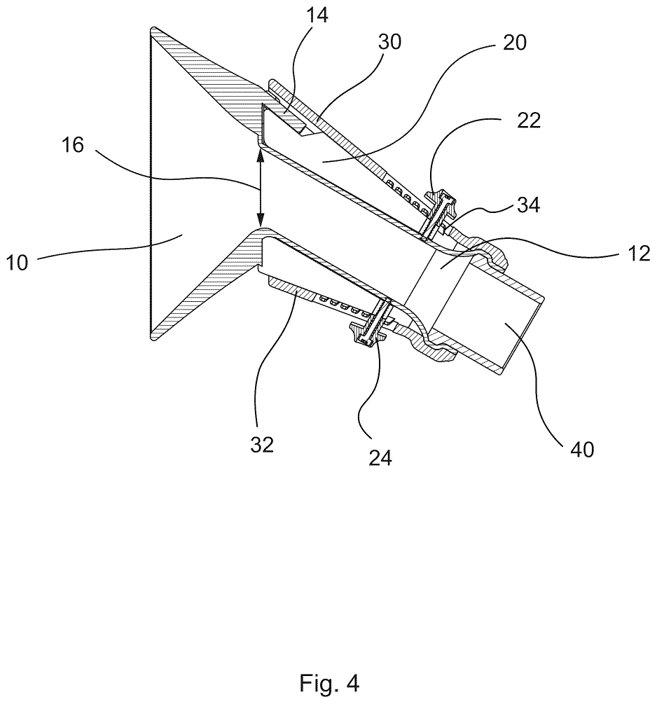

FIG. 4 provides a side sectional view of the collet 20, the button A 22, and the button B 24 in 15 mm nipple diameter configuration. When the numbers 36 position of button A 22 and button B 24 is adjusted distally from the cone, the frame A and frame B 32 cause the collet 20 diameter to shrink, radially compressing the opening 16 diameter. When the button A 22 and button B 24 are set to the numbers 36 15 mm position the collet may be configured to cause the opening 16 to be 18 mm, as shown in FIG. 4.

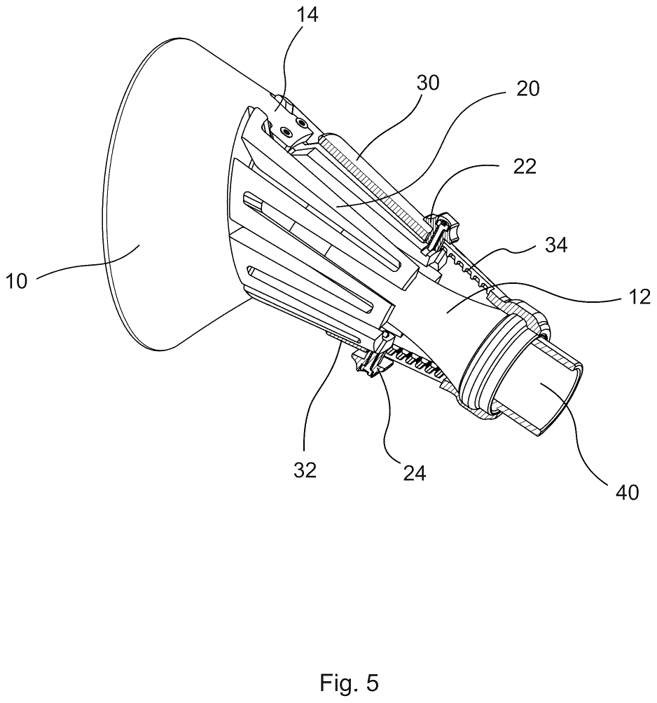

FIG. 5 provides an isometric partial cut view that shows the flange assembly with the collet 20, the button A 22, and the button B 24 in the 20 mm nipple diameter configuration, having the frame A 30, the frame B 32 and the cap 40 shown in cut view. The cone 10 is attached to the collet 20 with the tab 14. Preferably, the tab 14 is a flexible, clear LSR silicone material that can stretch to allow the opening 16 and the tube 12 to be centered axially in the collet 20 while the collet 20 changes in internal diameter. The section of the tube that is distal to the cone is compressed in between both the clear polypropylene frame A 30 and the frame B 32 on the outside and the clear polypropylene cap 40 on the inside. The compressed tube 12 section acts as a seal between the top cap and the tube to prevent milk from leaking out of the flange assembly.

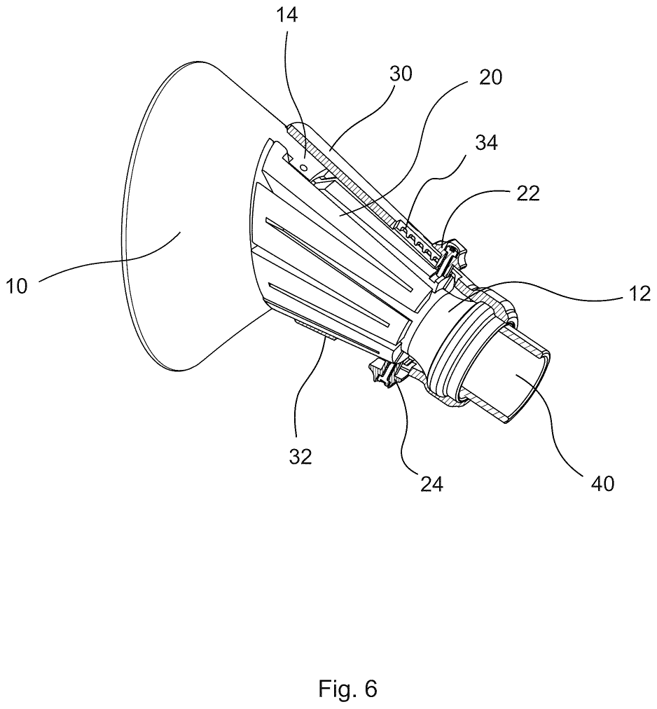

FIG. 6 provides an isometric partial cut view that shows the flange assembly with the collet 20, the button A 22, and the button B 24 in the 15 mm nipple diameter configuration, having the frame A 30, the frame B 32 shown in cut view.

Preferably, the cone 10, the tube 12, are made substantially of a flexible, optically clear or transparent, dishwasher-safe LSR Silicone material, for example silicone rubber. The substantially flexible silicone material of the cone 10 helps it conform to the shape of the breast, ensuring a tight seal around the nipple and areola. This cone 10 adaptability helps to prevent air leaks, maximizing milk expression. The substantially flexible nature of the cone material also minimizes skin discomfort and irritation, especially during extended pumping sessions.

Preferably, the transparent polypropylene or polyacetal or PEEK or polyphenysulfone collet 20, the frame 30, and the frame 32 are substantially made of a rigid, dishwasher safe material such as polypropylene, polycarbonate, nylon or another co-polymer. The cone 10, tube 12, collet 20, frame A 30, and frame B 32 are made substantially of a clear or transparent material to provide a view of the nipple and the inside of the cone. Transparent components such as the LSR Silicone cone 10 can help the user achieve correct nipple alignment when placing the flange assembly onto the breast. Dishwasher-safe components are important because the flange assembly should be cleaned and sterilized regularly and it is convenient for this to be accomplished in a normal dishwashing cycle.

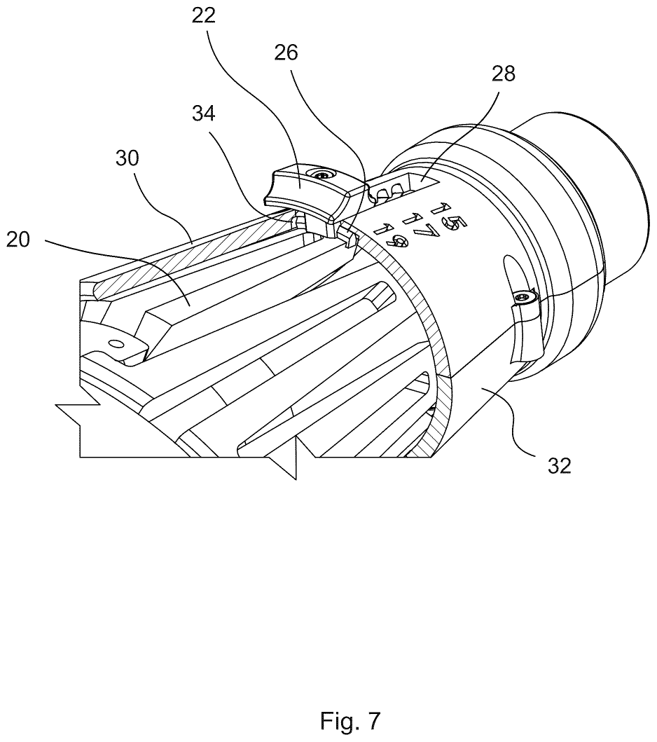

FIG. 7 provides a partial isometric cut view of the flange assembly having frame A 30 and Frame B 32 shown in cut view. The button A 22 and button B 24 are easily adjusted by the user by pressing and sliding the polypropylene buttons proximally or distally to the cone. When the buttons are released, the elastic force created by the collet 20 presses the button detent 26 into the frame detent pocket 34 to removably-fix the collet securely in place. This adjustment mechanism provides a quick, easy, and robust way to change the nipple diameter configuration of the flange assembly.

FIG. 7 further shows the button detent 26 removably-fixed into the frame detent pocket 34 in the (marked) 20 mm nipple diameter position/configuration.

### Further Alternate Embodiment(s)

Another way to describe the herein-disclosed invention is:
A breast pump flange assembly for extracting breast milk featuring an adjustable inner diameter for various nipple sizes, comprising a cone, a top cap, a compressed tube, a silicone funnel that contacts skin, a grippable frame, an adjustable flange assembly comprising a collet that constricts a silicone funnel to specific predetermined diameters, and at least two push buttons that adjust the collet to a diameter of between about 14 mm to about 20 mm.

The invention wherein the cone and the compressed tube are comprised of a clear, flexible LSR Silicone and the frame is comprised of a substantially clear polypropylene material.

The invention wherein the compressed tube acts as a seal between the top cap and the compressed tube to prevent milk from leaking out of the flange assembly.

The invention wherein the collet adjusts to be fixed at precisely a 14 mm diameter.

The invention wherein the collet adjusts to be fixed at precisely a 15 mm diameter.

The invention wherein the collet adjusts to be fixed at precisely a 16 mm diameter.

The invention wherein the collet adjusts to be fixed at precisely 17 mm diameter.

The invention wherein the collet adjusts to be fixed at precisely 17 mm diameter. The invention wherein the collet adjusts to be fixed at precisely a 18 mm diameter.

The invention wherein the collet adjusts to be fixed at precisely a 19 mm diameter.

The invention wherein the collet adjusts to be fixed at precisely a 20 mm diameter.

The invention wherein the collet adjustment mechanism facilitates a cone opening of between about 18 mm to about 23 mm.

An adjustable breast pump funnel assembly that engages with a breast pump to provide a tailored fit for various nipple sizes, comprising an adjustable collet, at least two button detents, a handling frame, a frame detent pocket, a substantially clear silicone adjustable funnel allowing inspection of nipple alignment, a flexible collet which manipulates the silicone funnel's diameter, and at least two push-sliding buttons which move along a grooved slot to translate and lock the collet at flange diameters of between about 14 mm and about 20 mm.

The invention wherein, when the buttons are pressed then slide along its groove and released, the collet pushes the button detent into the frame detent pocket, removably-fixing the collet in place, creating a leak-proof flange assembly that prevents milk-leakage, and further allowing the collet to be readjusted and reset by repeating the identical button manipulation.

A breast pump flange system comprising of an adjustment mechanism the provides a secure fit for a range of nipple sizes, comprising a skin-safe silicone funnel cone that provides a seal and transport for breast milk, a frame used for gripping and operating the device, a flange assembly comprising at least a first button and a second button, wherein the buttons are configured to slide against a funnel cone, wherein the cone is connected to a flexible collet comprising a detent mechanism that removably fixes the button in place at a selected nipple diameter configuration.

The invention wherein the collet mechanism is adjusted to an inner diameter of between about 4 mm and about 20 mm.

An adjustable breast pump flange assembly comprising an inner diameter that can be adjusted to accommodate various breast sizes without the need for additional inserts, wherein the flange comprises a collet mechanism that allows for precise diameter adjustment through a click-and-groove system, wherein the adjustable collet system eliminates the need for separate silicone inserts to match fixed nipple sizes, and wherein the flange assembly is composed of soft, transparent silicone.

### Specifications Generally

In the Specifications and in this Detailed Description, and the Claims below, and in the accompanying drawings, reference is made to particular features of various embodiments of the invention. It is to be understood that the disclosure of embodiments of the invention in this specification includes all possible combinations of such particular features. For example, where a particular feature is disclosed in the context of a particular aspect or embodiment of the invention, or a particular claim, that feature can also be used-to the extent possible-in combination with and/or in the context of other particular aspects and embodiments of the invention, and in the invention generally.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

In the present disclosure, various features may be described as being optional, for example, through the use of the verb “may;”, or, through the use of any of the phrases: “in some embodiments,” “in some implementations,” “in some designs,” “in various embodiments,” “in various implementations,”, “in various designs,” “in an illustrative example,” or “for example;” or, through the use of parentheses. For the sake of brevity and legibility, the present disclosure does not explicitly recite each and every permutation that may be obtained by choosing from the set of optional features. However, the present disclosure is to be interpreted as explicitly disclosing all such permutations. For example, a system described as having three optional features may be embodied in seven different ways, namely with just one of the three possible features, with any two of the three possible features or with all three of the three possible features.

In various embodiments, elements described herein as coupled or connected may have an effectual relationship realizable by a direct connection or indirectly with one or more other intervening elements.

In the present disclosure, the term “any” may be understood as designating any number of the respective elements, i.e. as designating one, at least one, at least two, each or all of the respective elements. Similarly, the term “any” may be understood as designating any collection(s) of the respective elements, i.e. as designating one or more collections of the respective elements, a collection comprising one, at least one, at least two, each or all of the respective elements. The respective collections need not comprise the same number of elements.

While various embodiments of the present invention have been disclosed and described in detail herein, it will be apparent to those skilled in the art that various changes may be made to the configuration, operation and form of the invention without departing from the spirit and scope thereof. In particular, it is noted that the respective features of embodiments of the invention, even those disclosed solely in combination with other features of embodiments of the invention, may be combined in any configuration excepting those readily apparent to the person skilled in the art as nonsensical. Likewise, use of the singular and plural is solely for the sake of illustration and is not to be interpreted as limiting.

In the present disclosure, all embodiments where “comprising” is used may have as alternatives “consisting essentially of,” or “consisting of” In the present disclosure, any method or apparatus embodiment may be devoid of one or more process steps or components. In the present disclosure, embodiments employing negative limitations are expressly disclosed and considered a part of this disclosure.

Certain terminology and derivations thereof may be used in the present disclosure for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “comprises” and grammatical equivalents thereof are used herein to mean that other components, ingredients, steps, among others, are optionally present. For example, an embodiment “comprising” (or “which comprises”) components A, Band C can consist of (i.e., contain only) components A, Band C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least I” means 1 or more than 1. The term “at most” followed by a number (which may be a range having 1 or Or as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)−(a second number),” this means a range whose limit is the second number. For example, 25 to 100 mm means a range whose lower limit is 25 mm and upper limit is 100 mm.

Many suitable methods and corresponding materials to make each of the individual parts of embodiment apparatus are known in the art. According to an embodiment of the present invention, one or more of the parts may be formed by machining, 3D printing (also known as “additive” manufacturing), CNC machined parts (also known as “subtractive” manufacturing), and injection molding, as will be apparent to a person of ordinary skill in the art. Metals, wood, thermoplastic and thermosetting polymers, resins and elastomers as may be described hereinabove may be used. Many suitable materials are known and available and can be selected and mixed depending on desired strength and flexibility, preferred manufacturing method and particular use, as will be apparent to a person of ordinary skill in the art.

Any element in a claim herein that does not explicitly state “means for” performing a specified function, or “step for” performing a specific function, is not to be interpreted as a “means” or “step” clause as specified in 35 U.S.C. § 112 (f). Specifically, any use of “step of in the claims herein is not intended to invoke the provisions of 35 U.S.C. § 112 (f). Elements recited in means-plus-function format are intended to be construed in accordance with 35 USC § 351 112 (f).

Recitation in a claim of the term “first” with respect to a feature or element does not necessarily imply the existence of a second or additional such feature or element.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.”

Any embodiment described herein as “exemplary” is not necessarily to be construed as preferred or advantageous over other embodiments. While the various aspects of the embodiments are presented in drawings, the drawings are not necessarily drawn to scale unless specifically indicated.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own.

1. A breast pump flange assembly for extracting breast milk featuring an adjustable inner diameter for various nipple sizes, comprising
a cone,
a top cap,
a compressed tube,
a silicone funnel that contacts
skin, a grippable frame,
an adjustable flange assembly comprising a collet that constricts a silicone funnel to specific predetermined diameters, and
at least two push buttons that adjust the collet to a diameter of between about 14 mm to about 20 mm.

2. The invention of claim 1 wherein the cone and the compressed tube are comprised of a clear, flexible LSR Silicone and the frame is comprised of a substantially clear polypropylene material.

3. The invention of claim 1 wherein the compressed tube acts as a seal between the top cap and the compressed tube to prevent milk from leaking out of the flange assembly.

4. The invention of claim 1 wherein the collet adjusts to be fixed at precisely a 14 mm diameter.

5. The invention of claim 1 wherein the collet adjusts to be fixed at precisely a 15 mm diameter.

6. The invention of claim 1 wherein the collet adjusts to be fixed at precisely a 16 mm diameter.

7. The invention of claim 1 wherein the collet adjusts to be fixed at precisely 17 mm diameter.

8. The invention of claim 1 wherein the collet adjusts to be fixed at precisely a 18 mm diameter.

9. The invention of claim 1 wherein the collet adjusts to be fixed at precisely a 19 mm diameter.

10. The invention of claim 1 wherein the collet adjusts to be fixed at precisely a 20 mm diameter.

11. The invention of claim 1 wherein the collet adjustment mechanism facilitates a cone opening of between about 18 mm to about 23 mm.

12. An adjustable breast pump funnel assembly that engages with a breast pump to provide a tailored fit for various nipple sizes, comprising
an adjustable collet,
at least two button
detents, a handling
frame,
a frame detent pocket,
a substantially clear silicone adjustable funnel allowing inspection of nipple alignment, a flexible collet which manipulates the silicone funnel's diameter,
and at least two push-sliding buttons which move along a grooved slot to translate and lock the collet at flange diameters of between about 14 mm and about 20 mm.

13. The invention of claim 12 wherein, when the buttons are pressed then slide along its groove and released, the collet pushes the button detent into the frame detent pocket, removably-fixing the collet in place, creating a leak-proof flange assembly that prevents milk-leakage, and further allowing the collet to be readjusted and reset by repeating the identical button manipulation.

14. A breast pump flange system comprising of an adjustment mechanism the provides a secure fit for a range of nipple sizes, comprising
a skin-safe silicone funnel cone that provides a seal and transport for breast milk, a frame used for gripping and operating the device,
a flange assembly comprising at least a first button and a second button, wherein the buttons are configured to slide against a funnel cone,
wherein the cone is connected to a flexible collet comprising a detent mechanism that removably fixes the button in place at a selected nipple diameter configuration, such that the adjustable breast pump flange assembly comprises an inner diameter that can be adjusted to accommodate various breast sizes without the need for additional inserts,
wherein the flange comprises a collet mechanism that allows for precise diameter adjustment through a click-and-groove system, and
wherein the adjustable collet system eliminates the need for separate silicone inserts to match
fixed nipple sizes, and
wherein the flange assembly is composed of soft, transparent silicone.

15. The invention of claim 14 wherein the collet mechanism is adjusted to an inner diameter of between about 14 mm and about 20 mm.
